(19) United States
(12) Patent Application Publication
(10) Pub. No.: US 2025/0135142 A1
(43) Pub. Date: May 1, 2025

(54) NASAL CANNULA COVER

(71) Applicant: TENSION SQUARE, LLC, Port Charlotte, FL (US)

(72) Inventors: Mychael Arnell OVERSTREET, Port Charlotte, FL (US); Sonya Yvonne OVERSTREET, Harker Heights, TX (US)

(21) Appl. No.: 18/930,398

(22) Filed: Oct. 29, 2024

Related U.S. Application Data
(60) Provisional application No. 63/546,232, filed on Oct. 29, 2023.

(51) Int. Cl. A61M 16/06; B33Y 80/00

(52) U.S. Cl. CPC A61M 16/0672; A61M 2209/06; B33Y 80/00

(57) ABSTRACT

A nasal cannula cover comprising two cooperative portions which may be when not in use “snapped together” while one portion remains moveably affixed to the cannula and moveably stored just above “a plastic cuff” commonly used to tighten/loosen the cannula. In certain embodiments, the nasal cannula cover can be attached to a user's ears.

### CROSS REFERENCE TO RELATED APPLICATION

This application claims the benefit of priority of U.S. provisional patent application No. 63/546,232, filed Oct. 29, 2023, the contents of which are all incorporated herein by reference in their entirety.

### FIELD OF THE INVENTION

The instant invention relates to a protective cover for use with nasal cannulas.

### BACKGROUND OF THE INVENTION

Respiratory interfaces, e.g., nasal cannulas are used to deliver respiratory gases for therapeutic effect, including oxygen therapy and other respiratory support. Small nasal cannulas are commonly used for delivery of low volumes of oxygen and other medical gasses to a user's nasal passage through a user's nostrils.

For patients switching to portable oxygen or in the case of home oxygen administration, nasal cannula storage can be problematic. Cannula prongs often become contaminated when patients don't properly protect the cannula between uses (i.e., leaving the cannula on the floor, furniture, bed linens, and the like). Consequently, a patient places the contaminated cannula back in their nostrils; thus, directly transferring potentially pathogenic organisms from these unsanitary surfaces into a patient's nostrils and the mucous membranes of their nasal passages; whereby placing themselves at risk of developing respiratory infections and the like.

Accordingly, there is a need in the art for an easy to produce and use nasal cannula cover method and system to protect the cannula when not is use; be easily and removably affixed to the cannula; and be captive to the cannula tubing when not in use.

### SUMMARY OF THE INVENTION

An object of the instant invention is to provide a nasal cannula cover comprising two cooperative portions which may be when not in use “snapped together” while one portion remains moveably affixed to the cannula and moveably stored just above “a plastic cuff” commonly used to tighten/loosen the cannula.

An object of the instant invention is to provide a nasal cannula cover comprising two separate and cooperative portions including precision cut-outs to facilitate a user removably affixing the cover to a nasal cannula and to removably affix a first portion of the cover to a second portion of the cover about the cannula nasal outlets; thereby protecting the nasal cannula from contamination when the nasal cannula is not in use.

An object of the instant invention is to provide a nasal cannula cover wherein the cover is made of a natural material.

An object of the instant invention is to provide a nasal cannula cover wherein the cover is made of a synthetic material.

An object of the instant invention is to provide a nasal cannula cover, wherein the cover is produced using additive manufacturing processes such as three-dimensional printing, injection molding.

An object of the instant invention is to provide a nasal cannula cover wherein the cover comprises an impact and crush resistant material able to bear loads of 250 pounds

An object of the instant invention is to provide a nasal cannula cover wherein the cover comprises a material which can survive impact acceleration of above 20 G's; and capable of surviving without operational damage being stepped on, being rolled over by a fully loaded wheelchair and/or gurney wheel.

An object of the instant invention is to provide a nasal cannula cover wherein the two portions may snap or screw together and attach to either side of cannula nasal outlet ports or posts.

An object of the instant invention is to provide a nasal cannula cover wherein the cover comprises a material capable of precision cutouts allowing for easy attachment to and removal from nasal cannulas and nasal cannula tubing.

An object of the instant invention is to provide a wherein when the cover is not in use; the two portions may can be snapped or screwed together vertically while still attached to a cannula and be stored just above commonly used “plastic cuffs” which tighten/loosen the cannula to the user's ears; preventing the two portions from rattling, sliding back and forth along the tubing during movement, and getting caught on objects during everyday activities; and from being misplaced and lost.

An object of the invention is to provide a nasal cannula cover that is capable of being compressed, i.e. stepped on or rolled over by wheelchair etc, and not breaking.

An object of the invention is to provide a nasal cannula cover that is 3D printed in impact resistant Nylon 11 or injection molded.

An object of the invention is to provide a nasal cannula cover that can bear a load of 250 lbs at its center and half an inch from center in either direction.

An object of the invention is to provide a nasal cannula cover that is formed from two halves that snap or screw together; the cover attaches to either side of cannula nose piece.

An object of the invention is to provide a nasal cannula cover that has precise cutouts allow for easy attachment to cannula tubing.

An object of the invention is to provide a nasal cannula cover that when not in use the two halves can be snapped together vertically, while still attached to cannula, and stored just above the plastic cuff used to tighten/loosen the cannula to the user's ears. This stops the two halves from rattling, sliding back and forth along the tubing during movement, and getting caught on objects during everyday activities.

Objects of the invention and its particular features and advantages will become more apparent from consideration of the following drawings and accompanying detailed description. Variations from the description above may and can be made by one skilled in the art without departing from the scope of the invention. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIGS. 1-7 show various views of the nasal cannula cover.

FIGS. 8-13 show screenshots of the two pieces of the nasal cannula cover being attached to one another.

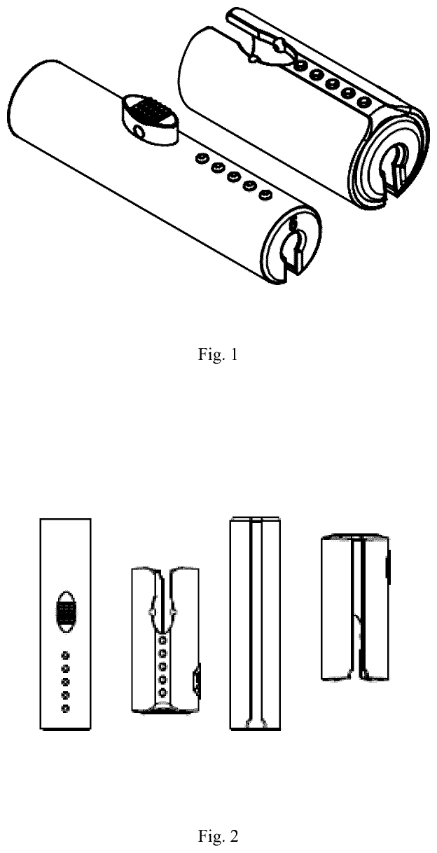

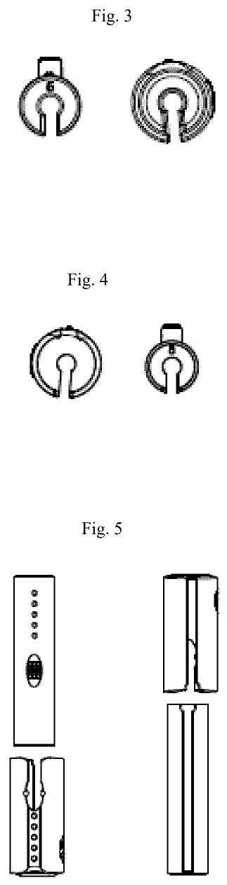

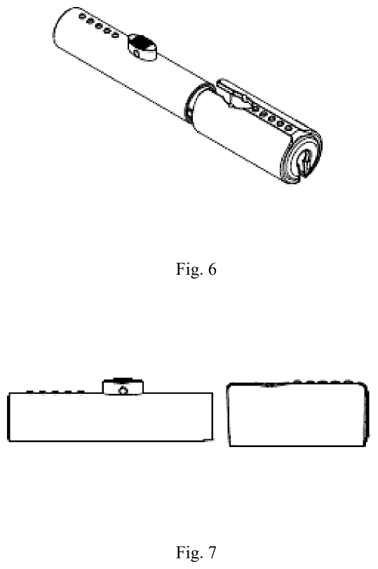

### DETAILED DESCRIPTION OF THE INVENTION

In the following description, numerous details are set forth for the purpose of example and explanation; however, one of ordinary skill in the art will realize that the invention may be practiced without the use of these specific details.

The instant inventive configuration prevents the two portions from rattling, sliding back and forth along the cannula tubing during patient movement, and prevents the two portions from getting snagged or caught on objects during daily activities.

As depicted in Figures below; the structure and configuration of the instant inventive nasal cannula cover can be easily appreciated.

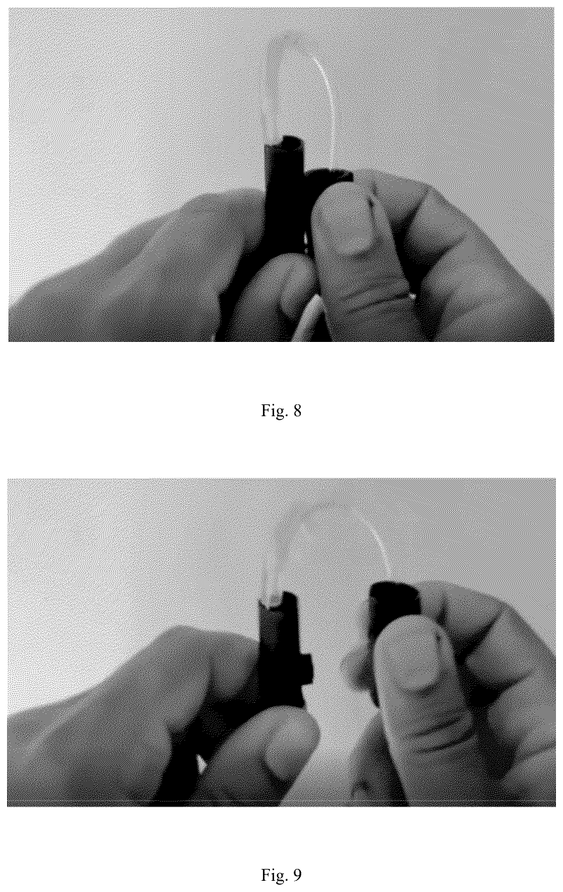

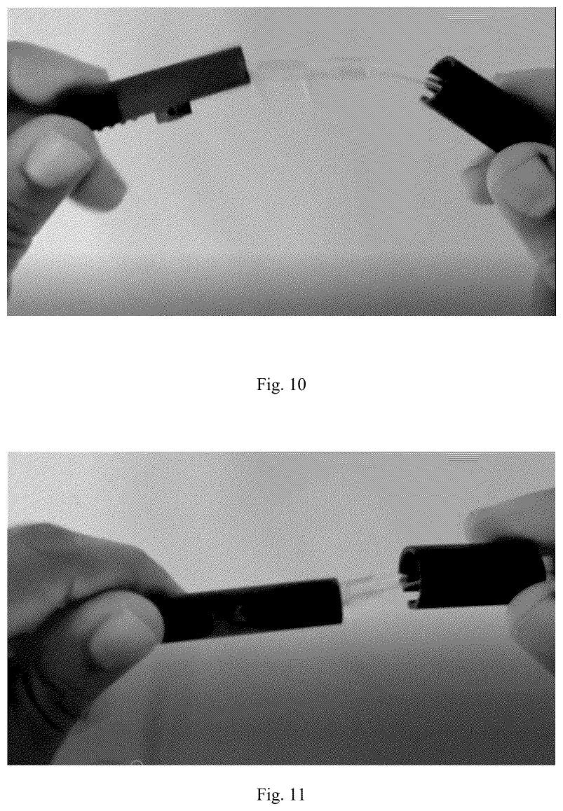

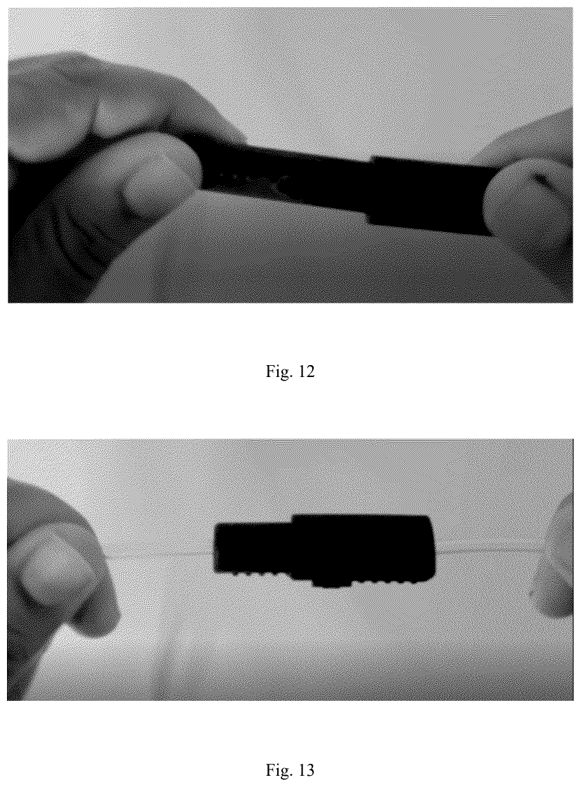

Additionally; the operation of the instant inventive nasal cannula cover may be easily appreciated from the follow “screen shots” on FIGS. 8-13 taken from an instant invention demonstrative video.

It is contemplated that the instant inventive nasal cannula cover comprises a natural material.

It is contemplated that the instant inventive nasal cannula cover comprises a synthetic material.

It is contemplated that the instant inventive nasal cannula cover be produced using additive manufacturing processes such as three-dimensional printing, injection molding, and the like.

It is contemplated that the instant inventive nasal cannula cover comprises an impact and crush resistant material able to bear loads of 250 pounds and which instant impact acceleration capability of above 20 G's; and capable of surviving without operational damage being stepped on, being rolled over by a fully loaded wheelchair and/or gurney wheel, and the like.

It is contemplated that the instant inventive nasal cannula cover comprises two portions which may snap or screw together and attach to either side of cannula nasal outlet ports or posts.

It is contemplated that the instant inventive nasal cannula cover comprises material capable of precision cutouts allowing for easy attachment to and removal from nasal cannulas and nasal cannula tubing.

It is contemplated that when the instant inventive nasal cannula cover is not in use; the two portions may can be snapped or screwed together vertically while still attached to a cannula and be stored just above commonly used “plastic cuffs” which tighten/loosen the cannula to the user's ears; which prevents the two portions from rattling, sliding back and forth along the tubing during movement, and getting caught on objects during everyday activities; and from being misplaced and lost.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

1. A nasal cannula cover comprising:
two separate and cooperative portions including precision cut-outs to facilitate a user removably affixing the cover to a nasal cannula and to removably affix a first portion of the cover to a second portion of the cover about the cannula nasal outlets;
thereby protecting the nasal cannula from contamination when the nasal cannula is not in use.

2. The nasal cannula cover of claim 1, wherein the cover is made of a natural material.

3. The nasal cannula cover of claim 1, wherein the cover is made of a synthetic material.

4. The nasal cannula cover of claim 1, wherein the cover is produced using additive manufacturing processes such as three-dimensional printing, injection molding.

5. The nasal cannula cover of claim 1, wherein the cover comprises an impact and crush resistant material able to bear loads of 250 pounds.

6. The nasal cannula cover of claim 1, wherein the cover comprises a material which can survive impact acceleration of above 20 G's; and capable of surviving without operational damage being stepped on, being rolled over by a fully loaded wheelchair and/or gurney wheel.

7. The nasal cannula cover of claim 1, wherein the two portions may snap or screw together and attach to either side of cannula nasal outlet ports or posts.

8. The nasal cannula cover of claim 1, wherein the cover comprises a material capable of precision cutouts allowing for easy attachment to and removal from nasal cannulas and nasal cannula tubing.

9. The nasal cannula cover of claim 1, wherein when the cover is not in use; the two portions may can be snapped or screwed together vertically while still attached to a cannula and be stored just above commonly used “plastic cuffs” which tighten/loosen the cannula to the user's ears; preventing the two portions from rattling, sliding back and forth along the tubing during movement, and getting caught on objects during everyday activities; and from being misplaced and lost.
